(19) United States
(12) Patent Application Publication
(10) Pub. No.: US 2025/0352563 A1
(43) Pub. Date: Nov. 20, 2025

(54) Method and Use of Psilocybin in the Prevention and Treatment of Stroke

(71) Applicant: Revive Therapeutics Ltd, Toronto (CA)

(72) Inventor: Fabio Chianelli, Toronto (CA)

(73) Assignee: Revive Therapeutics Ltd, Toronto (CA)

(21) Appl. No.: 18/862,882

(22) PCT Filed: May 2, 2023
(86) PCT No.: PCT/CA2023/050596

§ 371 Date: Nov. 4, 2024

Related U.S. Application Data
(60) Provisional application No. 63/337,704, filed on May 3, 2022.

(51) Int. Cl. A61K 31/675; A61P 9/10

(52) U.S. Cl. CPC A61K 31/675; A61P 9/10

(57) ABSTRACT

Methods for the prevention and treatment of stroke in a mammal including administering a therapeutically effective amount of psilocybin or a pharmaceutically acceptable salt or solvate thereof, to a mammal in need thereof. Use of a pharmaceutical composition including psilocybin or a pharmaceutically acceptable salt or solvate thereof, together with one or more pharmaceutically acceptable carriers, diluents and excipients for the prevention and treatment of stroke in a mammal.

### FIELD

The present invention relates to pharmaceutical compositions comprising psilocybin and their use for the prevention and treatment of stroke.

### BACKGROUND

Psilocybin (4-phosphoryloxy-N,N-dimethyltryptamine) is a substituted indolealkylamine and belongs to the group of hallucinogenic tryptamines. Psilocybin is a prodrug and undergoes dephosphorlyation to Psilocin in vivo. The chemical formulas for Psilocybin and Psilocin are:

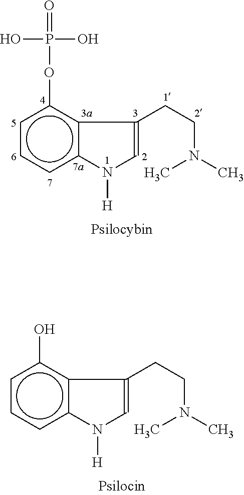

Psilocybin was distributed worldwide under the name Indocybin® (Sandoz) as a short-acting and more compatible substance (than, for example, LSD) to support is use as a psychotherapeutic. Experimental and therapeutic use was extensive and without complications. (1)

Stroke is a major leading cause of death and adult disability. Various pharmacological agents, including 5-HT2R agonists, have been examined for the protective response against stroke. For example, in a rat model of stroke, intraperitoneal injection of DMT prior to the middle cerebral artery occlusion (MCAo) improved staircase behavior, increased BDNF expression, and reduced brain inflammation and infarction (18). However, the protective effect of psilocybin in stroke models has not been investigated.

Any discussion of documents, acts, materials, devices, articles or the like which has been included in the present specification is not to be taken as an admission that any or all of these matters form part of the prior art base or were common general knowledge in the field relevant to the present invention as it existed before the priority date of each claim of this application.

### SUMMARY OF THE INVENTION

The present disclosure, in one aspect, relates to a method for the prevention and treatment of stroke in a mammal comprising administering a therapeutically effective amount of psilocybin or a pharmaceutically acceptable salt or solvate thereof, to a mammal in need thereof. In one embodiment, the stroke is ischemic brain injury. In certain embodiments, the therapeutically effective amount of psilocybin or a pharmaceutically acceptable salt or solvate thereof is administered pre-stroke or early post-stroke.

The present disclosure, in another aspect, relates to a use of a pharmaceutical composition including psilocybin or a pharmaceutically acceptable salt or solvate thereof, together with one or more pharmaceutically acceptable carriers, diluents and excipients for the prevention and treatment of a stroke. In one embodiment, the stroke is ischemic brain injury. In certain embodiments, the therapeutically effective amount of psilocybin or a pharmaceutically acceptable salt or solvate thereof is administered pre-stroke or early post-stroke.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 Psilocybin significantly reduced glutamate-mediated neuronal loss in rat primary cortical neuronal culture. (A) are representing photomicrographs which indicate that Glutamate (Glu, 100 μM) reduced the neuronal marker MAP2-ir. Glu-mediated neuronal loss was antagonized by Psilocybin (Psi, 10 μM). (B) is a photomicrograph and a bar graph of MAP2 immunoreactivity. MAP2-ir from all studies was averaged and analyzed analyzed (veh=6; Glu, n=12; Glu+Psi 0.1 μM, n=12); Glu+Psi 1 μM, n=4; Glu +Psi 10 μM, n=6). Glu significantly reduced MAP2-ir (p<0.001). Psi dose-dependently mitigated this response (p<0.05, one-way ANOVA+NK test). (C) is a schematic diagram of a timeline of an in vivo experiment;

FIG. 2 Pretreatment with Psilocybin reduced behavioral deficits in stroke rats. (A) Timeline of the in vivo study. Psilocybin (Psi) or vehicle (veh) was given (i.c.v.) at 10-15 min prior to the MCAo on D0. (B) Neurological and (C) locomotor tests were conducted on D2. Pretreatment with Psi significantly decreased (B1) Bederson's neurological score, (B2) body asymmetry, while improved (C1) horizontal activity (HACTV), (C2) total distance traveled (TOTDIST), (C3) movement number (MOVNO), and (C4) movement time (MOVETIME). N=7 per group;

FIG. 3 Pretreatment with Psilocybin reduced brain infarction in stroke rats. (A) Representing TTC images from 2-mm brain slices taken from stroke rats. White regions represent the area of infarction. Psilocybin (Psi; i.c.v.) reduced the infarct volume. (B) The area of infarction per each 2-mm slice from rostral to caudal (1 to 7) was quantified for all animals (n=7 per group). Psi significantly reduced infarction (*p<0.001, the difference between Psi and veh; #p<0.05, difference in slices, Two-way ANOVA+NK test);

FIG. 4 Early post-treatment with Psilocybin improved behavior function in stroke rats. (A) Timeline of in vivo study. Psilocybin (Psi, 1 mg/kg/d, n=7) or vehicle (n=6) was given (i.p.) daily between days 3 and 7 after the MCAo. Neurological and locomotor tests were conducted on day 9. Psi post-treatment significantly reduced (B1) Bederson's neurological score and (B2) body asymmetry, while increasing locomotor behavior (C1-4: HACTV, TOTDIST, MOVNO, MOVTIME). p<0.05, t-test;

FIG. 5 Post-stroke treatment with Psilocybin improved the expression of neuronal markers and BDNF. Cerebral cortical tissue was collected from naive (n=6) and stroke rats receiving systemic Psilocybin (Psi, n=7) or veh (n=6). The expression of (A-D) MAP2, synaptophysin (SYP), IBA1, and BDNF was analyzed by qRTPCR. Psi significantly normalized the expression of MAP2, SYP, and IBA1 in the stroke brain (A-C, *p<0.05). (D) The expression of BDNF in the lesioned brain was upregulated by Psi (p=0.01). One-way ANOVA+NK-test;

FIG. 6 ANA12 antagonized Psilocybin-mediated protection in primary neuronal culture. (A) Representing MAP2 immunostaining demonstrates that Psi antagonized Glu-mediated neuronal loss. The improvement in MAP2-ir was mitigated by ANA12. (B) MAP2-ir was analyzed in all wells (n=6 per each group). ANA12 significantly antagonized Psilocybin (Psi)-mediated protection (Glu+Psi vs Glu 100 μM +Psi 10 μM +ANA 10 μM, #p<0.05, one-way ANOVA). (C) Timeline; and

ANA12 antagonized Psilocybin-mediated neuroprotection in brain infarction and behavior. (A) Timeline. Psi was given (i.c.v.) at 15 min prior to the MCAo. ANA12 or veh was given intranasally after the MCAo on days 0 and 1 (n=7 per each group). (B) ANA12 significantly antagonized Psilocybin (Psi)-mediated reduction in brain infarction and behavior improvements (C: neurological function; D: locomotor behavior).

### DETAILED DESCRIPTION

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

For use in therapy a therapeutically effective amount of the psilocybin or pharmaceutically acceptable salts or solvates thereof, may be presented as a pharmaceutical composition. Thus, in a further embodiment the invention provides a pharmaceutical composition of psilocybin or pharmaceutically acceptable salts or solvates thereof in admixture with one or more pharmaceutically acceptable carriers, diluents, or excipients. The carrier(s), diluent(s) or excipient(s) must be acceptable in the sense of being compatible with the other ingredients of the formulation and not deleterious to the recipient thereof.

When applicable, the compositions of the present invention, including psilocybin may be in the form of and/or may be administered as a pharmaceutically acceptable salt.

Typically, a pharmaceutically acceptable salt may be readily prepared by using a desired acid or base as appropriate. The salt may precipitate from solution and be collected by filtration or may be recovered by evaporation of the solvent.

Suitable addition salts are formed from acids which form non-toxic salts and examples are hydrochloride, hydrobromide, hydroiodide, sulphate, nitrate, phosphate, hydrogen phosphate, dihydrogen phosphate acetate, maleate, malate, fumarate, lactate, tartrate, citrate, formate, gluconate, succinate, pyruvate, oxalate, oxaloacetate, trifluoroacetate, saccharinate, benzoate, methanesulphonate, ethanesulphonate, benzenesulphonate, p-toluenesulphonate and isethionate.

Suitable salts may also be formed from bases, forming salts including ammonium salts, alkali metal salts such as those of sodium and potassium, alkaline earth metal salts such as those of calcium and magnesium.

Pharmaceutically acceptable salts may also be prepared from other salts, including other pharmaceutically acceptable salts, using conventional methods.

Those skilled in the art of organic or coordination chemistry will appreciate that many organic and coordination compounds can form complexes with solvents in which they are reacted or from which they are precipitated or crystallized. These complexes are known as “solvates”. For example, a complex with water is known as a “hydrate”. Solvates of psilocybin are within the scope of the present invention.

Pharmaceutical compositions of the invention may be formulated for administration by any appropriate route, for example by the oral (including buccal or sublingual). Therefore, the pharmaceutical compositions of the invention may be formulated, for example, as tablets, capsules, powders, granules, lozenges, creams or liquid preparations, such as oral solutions or suspensions. Such pharmaceutical formulations may be prepared by any method known in the art of pharmacy, for example by bringing into association the active ingredient with the carrier(s) or excipient(s).

Tablets and capsules for oral administration may be in unit dose presentation form, and may contain conventional excipients such as binding agents, for example syrup, acacia, gelatine, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatine, hydroxyethyl cellulose, carboxymethyl cellulose, aluminium stearate gel or hydrogenated edible fats, emulsifying agents, for example lecithin, sorbitan, monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

It should be understood that in addition to the ingredients particularly mentioned above, the formulations may include other agents conventional in the art having regard to the type of formulation in question.

The compositions of the present invention may be suitable for the treatment of diseases in a human or animal patient. In one embodiment, the patient is a mammal including a human, horse, dog, cat, sheep, cow, or primate. In one embodiment the patient is a human. In a further embodiment, the patient is not a human.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

In certain embodiments of the present invention, pharmaceutically acceptable compositions of the present disclosure can be administered to humans and other animals at doses within the range of about 0.5 mL/day to about 3.0 mL/day and at a concentration within the range of about 0.5 mM to about 3.0 mM, particularly within the range of about 0.5 mL/day to about 3.0 mL/day and at a concentration within the range of about 0.5 mM to about 1.0 mM, particularly within the range of about 0.5 mL/day to about 3.0 mL/day and at a concentration within the range of about 0.5 mM to about 1.5 mM, particularly within the range of about 0.5 mL/day to about 3.0 mL/day and at a concentration within the range of about 0.5 mM to about 2.0 mM, particularly within the range of about 0.5 mL/day to about 3.0 mL/day and at a concentration within the range of about 0.5 mM to about 2.5 mM, particularly within the range of about 0.5 mL/day to about 1.0 mL/day and at a concentration within the range of about 0.5 mM to about 1.0 mM, particularly within the range of about 0.5 mL/day to about 1.5 mL/day at a concentration within the range of about 0.5 mM to about 1.0 mM, particularly within the range of about 0.5 mL/day to about 2.0 mL/day at a concentration within the range of about 0.5 mM to about 1.0 mM, particularly within the range of about 0.5 mL/day to about 2.5 mL/day at a concentration within the range of about 0.5 mM to about 1.0 mM, particularly within the range of about 0.5 mL/day to about 3.0 mL/day at a concentration within the range of about 0.5 mM to about 1.0 mM, particularly within the range of about 0.5 mL/day to about 1.0 mL/day at a concentration within the range of about 0.5 mM to about 1.5 mM, particularly within the range of about 0.5 mL/day to about 1.0 mL/day at a concentration within the range of about 0.5 mM to about 1.5 mM, particularly within the range of about 0.5 mL/day to about 2.0 mL/day and at a concentration within the range of about 0.5 mM to about 1.5 mM, particularly within the range of about 0.5 mL/day to about 2.5 mL/day at a concentration within the range of about 0.5 mM to about 1.5 mM, particularly within the range of about 0.5 mL/day to about 3.0 mL/day at a concentration within the range of about 0.5 mM to about 1.5 mM, particularly within the range of about 0.5 mL/day to about 1.0 mL/day at a concentration within the range of about 0.5 mM to about 2.0 mM, particularly within the range of about 0.5 mL/day to about 1.5 mL/day and at a concentration within the range of about 0.5 mM to about 2.0 mM, particularly within the range of about 0.5 mL/day to about 2.0 mL/day at a concentration within the range of about 0.5 mM to about 2.0 mM, particularly within the range of about 0.5 mL/day to about 2.5 mL/day at a concentration within the range of about 0.5 mM to about 2.0 mM, particularly within the range of about 0.5 mL/day to about 3.0 mL/day and at a concentration within the range of about 0.5 mM to about 2.0 mM, particularly within the range of about 0.5 mL/day to about 1.0 mL/day at a concentration within the range of about 0.5 mM to about 2.0 mM, particularly within the range of about 0.5 mL/day to about 1.5 mL/day and at a concentration within the range of about 0.5 mM to about 2.0 mM, particularly within the range of about 0.5 mL/day to about 2.0 mL/day and at a concentration within the range of about 0.5 mM to about 2.0 mM, particularly within the range of about 0.5 mL/day to about 2.5 mL/day at a concentration within the range of about 0.5 mM to about 2.0 mM, particularly within the range of about 0.5 mL/day to about 3.0 mL/day and at a concentration within the range of about 0.5 mM to about 2.0 mM, particularly within the range of about 0.5 mL/day to about 1.0 mL/day and at a concentration within the range of about 0.5 mM to about 2.5 mM, particularly within the range of about 0.5 mL/day to about 1.5 mL/day at a concentration within the range of about 0.5 mM to about 2.5 mM, particularly within the range of about 0.5 mL/day to about 2.0 mL/day and at a concentration within the range of about 0.5 mM to about 2.5 mM, particularly within the range of about 0.5 mL/day to about 2.5 mL/day and at a concentration within the range of about 0.5 mM to about 2.5 mM, particularly within the range of about 0.5 mL/day to about 3.0 mL/day at a concentration within the range of about 0.5 mM to about 2.5 mM, particularly a dose of 0.5 mL/day at a 2 mM concentration, particularly a dose of 1.0 mL/day at a 2 mM concentration, particularly a dose of 1.2 mL/day at a 2 mM concentration, particularly a dose of 1.5 mL/day at a 2 mM concentration, particularly a dose of 2.0 mL/day at a 2 mM concentration, particularly a dose of 2.5 mL/day at a 2 mM concentration, particularly a dose of 3 mL/day at a 2 mM concentration, particularly a dose within the range of about 1.8 mL/day to about 2.2 mL/day at a concentration within the range of about 1.8 mM to about 2.2 mM, particularly a dose within the range of about 1.9 mL/day to about 2.1 mL/day at a concentration within the range of about 1.9 mM to about 2.1 mM, particularly within the range of about 0.5 mL/day to about 3.0 mL/day at a 2 mM concentration, particularly up to 3 mL/day at a concentration up to 3.0 mM, particularly up to 3 mL/day at a 2 mM concentration, and this should provide a therapeutically effective dose. However, the daily dose will necessarily be varied depending upon the host treated, the particular route of administration, and the severity of the illness being treated. Accordingly, the optimum dosage may be determined by the practitioner who is treating any particular patient. In another embodiment, the optimal dose may be higher.

As used herein the term “treatment” refers to defending against or inhibiting a symptom, treating a symptom, delaying the appearance of a symptom, reducing the severity of the development of a symptom, and/or reducing the number or type of symptoms suffered by an individual, as compared to not administering a pharmaceutical composition of the invention. The term treatment encompasses the use in a palliative setting.

### SUMMARY OF EXPERIMENTAL STUDY

A study was designed to evaluate the examine psilocybin-mediated protection in stroke rats. The protective effect of psilocybin in neuronal culture and in an animal model of stroke was characterized. Psilocybin reduced the glutamate-mediated loss of MAP2-ir in primary neuronal cultures. Parallel neuroprotective effects were seen in experimental animals. It was demonstrated that pre or early post-stroke treatment with Psilocybin significantly reduced neurological deficits, improved locomotor behavioral function, reduced brain infarction, and suppressed expression of inflammatory in stroke rats. The main finding of this study is that psilocybin is a potent neuroprotective agent against ischemic stroke in animals and mamals.

Two psilocybin regimens were used in this study. Psilocybin was given intracerebroventricularly prior to the MCAo as psilocybin is inefficient to freely cross the blood-brain barrier in non-lesioned brain (8, 22). In the post-stroke experiment, psilocybin was given intraperitoneally at 3 to 7 days after MCAo because the BBB was compromised shortly after stroke. In both studies, psilocybin treatment significantly reduced neurological deficits, as seen in the improvement in Bederson's neurological score and body asymmetry. Animals also showed significant improvement in locomotor activity. These data suggest that psilocybin, given pre-or early post-MCAo, normalizes motor behaviors in stroke rats.

Associated with the improvement in behavior, we demonstrated that psilocybin reduced brain infarction and improved the expression of neuronal markers (i.e., MAP2 and synaptophysin) in the lesioned brain. These data support that psilocybin antagonized neurodegenerative responses after stroke.

After the onset of stroke, progressive inflammatory reactions are activated and last for days, which lead to cell death and affect neural repair. These inflammatory responses include activation of innate microglia, overproduction of cytokines and chemokines, and infiltration of peripheral immune cells (23). We and others previously reported that suppression of inflammation through pharmacological agents reduces neurodegeneration and improves behavioral recovery from ischemic injury (19, 24, 25). In this study, post-treatment with psilocybin suppressed the expression of IBA1, supporting the anti-inflammatory role of psilocybin in stroke brain. This finding is further supported by the recent reports that the extracts of psilocybin-containing mushroom downregulated the expression of pro-inflammatory mediators (26, 27).

Increasing evidence support the neurotrophic role of BDNF in stroke brain. BDNF regulates neuroprotection, neuroplasticity, and repair after ischemic brain injury (28, 29). It was found that psilocybin increased the expression of BDNF in stroke brain. To further identify the role of BDNF in psilocybin-mediated protection, a BDNF specific antagonist ANA12 was used (30). In the in vitro cell culture, the present study demonstrated that ANA12 significantly antagonized psilocybin-mediated improvement in MAP2-ir. In the in vivo study, ANA12 significantly antagonized psilocybin-mediated improvements in brain infarction and locomotor behavior.

These data suggest that BDNF is involved in the protective action of psilocybin. In conclusion, the present study demonstrated that psilocybin is neuroprotective against excitatory amino acid and ischemia-mediated brain injury. Early posttreatment with psilocybin reduces neurodegeneration, inflammation, and neurological deficits in stroke animals. The mechanism of protection involves anti-inflammation and upregulation of protective neurotrophic factors. Psilocybin is currently under clinical trials for depression and anxiety (see clinicaltrials.gov). It has been reported that stroke patients have a higher incidence of developing depression and anxiety (31, 32). Psilocybin may be a useful therapeutic agent to prevent or treat comorbidity of stroke and depression in patients.

### Materials and Methods

### Animals and Drugs

Adult male and time-pregnant Sprague-Dawley rats were purchased from BioLASCO, Taipei, Taiwan. The use of animals was approved by the Animal Research Committee of the National Health Research Institutes of Taiwan (NHRI-IACUC-109097-A). All animal experiments were carried out in accordance with the National Institutes of Health Guide for the Care and Use of Laboratory Animals.

Psilocybin was purchased from the Cayman Chemical Company, Ann Arbor, Michigan, USA). The use of control substance Psilocybin for the in vivo and in vitro studies was approved by the Taiwan FDA (approval number GRR09600000107). ANA12 (SML0209) was purchased from the Sigma-Aldrich.

### Primary Cultures of Rat Cortical Neurons (PCN)

Primary cultures were prepared from embryonic (E14-15) cortex tissues obtained from fetuses of timed pregnant Sprague-Dawley rats. After removing the blood vessels and meninges, pooled cortices were trypsinized (0.05%; Invitrogen, Carlsbad, CA) for 20 min at room temperature. After rinsing off trypsin with pre-warmed Dulbecco's modified Eagle's medium (Invitrogen), cells were dissociated by trituration, counted, and plated into 96-well (5.0×104/well) cell culture plates precoated with Poly-D-Lysine (Sigma-Aldrich, St. Louis, MO). The culture plating medium consisted of neurobasal medium supplemented with 2% heat-inactivated fetal bovine serum (FBS), 0.5 mM L-glutamine, 0.025 mM L-glutamate and 2% B27 (Invitrogen). Cultures were maintained at 37° C. in a humidified atmosphere of 5% CO2 and 95% air. The cultures were fed by exchanging 50% of media with feed media (Neurobasal medium, Invitrogen) with 0.5 mM L-glutamate and 2% B27 with antioxidants supplement on days in vitro (DIV) 3 and 5. On DIV 7 and 10, cultures were fed with media containing B27 supplement without antioxidants (Invitrogen). On DIV 10, cultures were treated with reagents. After 48 hrs, cells were fixed 4% paraformaldehyde for 1 hour at room temperature.

### Immunocytochemistry

After removing 4% PFA solution, cells were washed with PBS and the fixed cells were treated for 1 h with a blocking solution (5% BSA and 0.1% Triton X-100 in PBS). The cells were then incubated for 1 day at 4° C. with a mouse monoclonal antibody against MAP2 (1:500; Millipore, Billerica, MA). The cells were then rinsed three times in PBS. The bound primary antibody was visualized using AlexaFluor 488 goat anti-mouse secondary (Invitrogen). Images were acquired using a camera DS-Qi2 (Nikon, Melville, NY) attached to a NIKON ECLIPSE Ti2 (Nikon, Melville, NY). Data were analyzed using NIS-Elements AR 5.11 Software (Nikon) and evaluated.

### Drug Delivery and Stroke Surgery

Psilocybin or vehicle was given before or after the MCAo. For the pretreatment, animals were anesthetized. Psilocybin (100 μM×20 μL, n=7) or vehicle (saline, 20 μl, n=7) was administered intracerebroventricularly (i.c.v., AP, −0.8 mm; LV, −1.5 mm; DV, −3.5 mm) at 15 min before MCAo through a Hamilton microsyringe. The speed of injection was controlled by a syringe pump (Micro 4, WPI, Sarasota, FL). A subgroup of animals were used to examine the interaction of Psilocybin and ANA12. These animals received Psilocybin (100 μM×20 μL, i.c.v.) at 15 min before MCAo.

ANA12 (100 μM×20 μL/d, n=7) or vehicle (20 μL/d, n=7) was given intranasally after the MCAo on days 0 and 1. In the posttreatment group, Psilocybin (1 mg/kg/day, n=7) or vehicle (n=6) was administered i.p. from day 3 to day 7. Animals were sacrificed on day 9 for PCR analysis.

The right middle cerebral artery (MCA) was transiently occluded (MCAo) as we previously described (33). In brief, the bilateral common carotids were clamped with nontraumatic arterial clips. The right MCA was ligated with a 10-O suture to generate focal infarction in the cerebral cortex. The ligature and clips were removed after 60 min ischemia to generate reperfusion injury. Core body temperature was monitored with a thermistor probe and maintained at 37° C. with a heating pad during anesthesia. After recovery from anesthesia, body temperature was maintained at 37° C. using a temperature-controlled incubator.

### Neurological Test

Two neurological tests were used to examine stroke behavior. (a) Body asymmetry was analyzed using an elevated body asymmetry test (19, 34). Rats were examined for lateral movements/turning when their bodies were suspended 20 cm above the testing table by lifting their tails. The frequency of initial turning of the head or upper body contralateral to the ischemic side was counted in 20 consecutive trials. The maximum impairment in body asymmetry in stroke animals is 20 contralateral turns/20 trials. In non-stroke rats, the average body asymmetry is 10 contralateral turns/20 trials (i.e., the animals turn in each direction with equal frequency). (b) Neurological deficits were scored by the Bederson's neurological test (35, 36). In a postural reflex test, rats were examined for the degree of abnormal posture when suspended by 20-30 cm above the testing table. They were scored according to the following criteria.
0 Rats extend both forelimbs straight. No observable deficit.
1 Rats keep the one forelimb to the breast and extend the other forelimb straight.
2 Rats show decreased resistance to lateral push in addition to behavior in score 1 without circling.
3 Rats twist the upper half of their body in addition to behavior in score 2.

### Locomotor Behavioral Measurement

Locomotion was measured using an infrared activity monitor (Accuscan, Columbus, OH) as we previously described (19, 37). Rats were individually placed in a 3D infrared behavior chamber (42×42×21 cm) for 60 min. Four parameters were recorded: (a) horizontal activity (HACTV, the total number of beam interruptions that occurred in the horizontal sensors), (b) total distance traveled (TOTDIST, the distance, in centimeters, traveled by the animals), and (c) No. of movements (MOVNO), (d) horizontal movements time (MOVTIME).

### Triphenyl Tetrazolium Chloride (TTC) Staining

The brains were removed, immersed in cold saline for 5 minutes, and sliced into 2.0 mm thick sections. The brain slices were incubated in 2% TTC (T8877, Sigma-Aldrich, MO, USA), dissolved in normal saline for 10 minutes at room temperature, and then transferred into a 5% formaldehyde solution for fixation. The area of infarction on each brain slice was measured double-blind using a digital scanner and the Image Tools program (University of Texas Health Sciences Center, San Antonio, TX).

### Quantitative Reverse Transcription PCR (qRT-PCR)

Cortex tissues from the lesioned side hemispheres were collected for qRPTCR analysis. Total RNAs were isolated using TRIzol Reagent (ThermoFisher, #15596-018), and cDNAs were synthesized from 1 μg total RNA using RevertAid H Minus First Strand cDNA Synthesis Kit (Thermo Scientific, #K1631). cDNA levels for IBA1, BDNF, MAP2, Synaptophysin (SYP), and GAPDH were determined by specific universal probe library primer-probe sets or gene-specific primers (Table 1). Samples were mixed with TaqMan Fast Advanced Master Mix (Life Technologies, #4444557) or SYBR (Luminaris Color HiGreen Low ROX qPCR Master Mix; ThermoScientific). Quantitative real-time PCR (qRT-PCR) was carried out using the QuantStudio™ 3 Real-Time PCR System (ThermoScientific). The expression of the target genes (Table 1) was normalized relative to the endogenous reference gene (GAPDH) with a modified delta-delta-Ct algorithm. All experiments were carried out in duplicate.

TABLE 1
Oligonucleotide primers used for quantitative RT-PCR.

| | SYBR Green | | |
|---|---|---|---|
| Gene | Forward | Reverse | TagMan |
| BDNF | CACTTTTGAGCACGTCATCG | TCCTTATGGTTTTCTTCGTTGG | |
| MAP2 | CAAACGTCATTACTTTACAACTTGA | CAGCTGCCTCTGTGAGTGAG | |
| SYP | TTGGCTTCGTGAAGGTGCTGCA | ACTCTCCGTCTTGTTGGCACAC | |
| IBA1 | | | Rn00574125_g1 |
| GAPDH | | | Rn01775763_g1 |

### Statistical Analysis

Data were presented as mean±s.e.m. Student's t-test, one or two-way ANOVA, and post-hoc Newman-Keuls test (NK test) were used for statistical comparisons. All analyses were calculated by Sigmaplot software version 10.0. Statistical significance was defined as p<0.05.

### Results

### Psilocybin Dose-Dependently Antagonized Glutamate Neurotoxicity in Primary Cortical Neuronal Culture

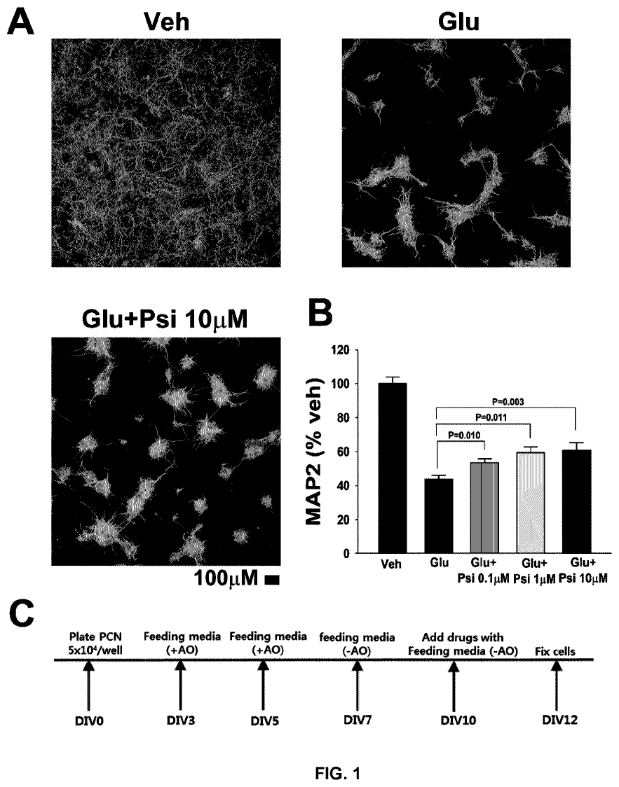

Glutamate (100 μM), Psilocybin (0.1, 1, 10 μM), or vehicle were added to the primary cortical culture (19) on DIV10 (see timeline in FIG. 1C). A high dose (100 μM) of glutamate was used to generate neurodegeneration and to simulate the overflow of elevated glutamate during cerebral ischemia (20, 21). Cells were fixed for Microtubule-Associated Protein 2 (MAP2) immunocytochemistry on DIV12. Glutamate significantly reduced MAP2 immunoreactivity (MAP-ir, FIG. 1A and B, p<0.001). Psilocybin dose-dependently mitigated this response (FIG. 1B, p<0.05, 1-Way ANOVA).

### Intracerebral Administration of Psilocybin Reduced Behavioral Deficits in Stroke Rats

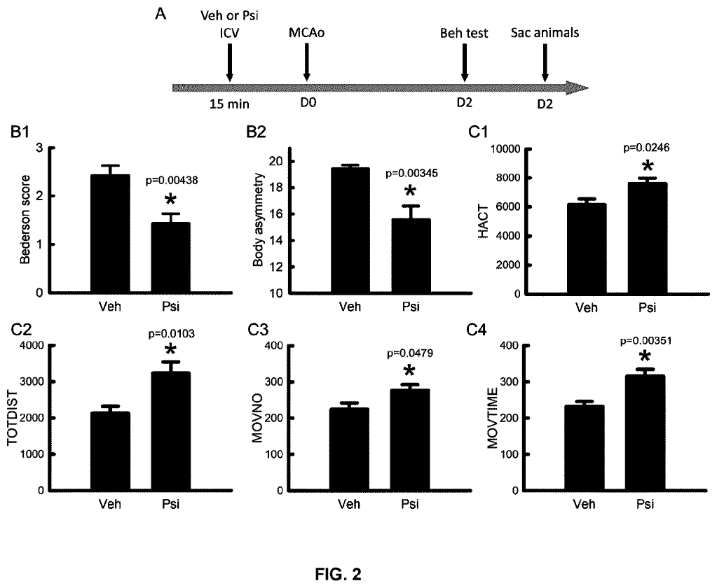

As psilocybin was neuroprotective in primary neuronal culture, we next examined if a similar protective response can be found in experimental stroke animals. A total of 14 adult rats were used for this analysis. Psilocybin (100 μM×20 μL, n=7) or vehicle (saline, 20 μL, n=7) was delivered to the lateral ventricle (i.c.v., coordinates: AP:0.8 mm, Lat:1.5 mm, DV −3.5 mm) at 10-15 min prior to the a 60-min MCAo. Behavioral tests were conducted two days (or D2) after MCAo (FIG. 2A). Pretreatment with Psilocybin significantly decreased Bederson's neurological score (FIG. 2-B1, p=0.00438, t-test) and body asymmetry (FIG. 2-B2, p=0.00345). Locomotoractivity was examined in infra-red activity chambers for one hour. Psilocybin, compared to vehicle, significantly increased horizontal activity (HACTV, p=0.0246), total distance traveled (TOTODIST, p=0.0103), movement number (MOVNO, p=0.0479), and movement time (MOVTIME, p=0.00351; FIG. 2C1-4). These data suggest that Psilocybin, given intracerebroventricularly, reduced neurological deficits and improved locomotor activity in stroke rats.

### Intracerebral Administration of Psilocybin Reduced Brain Infarction in Stroke Rats

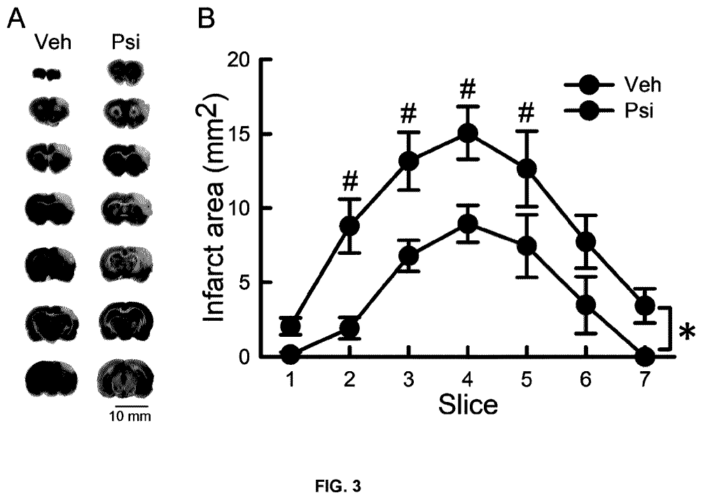

Animals were sacrificed for brain infarction measurement after the behavioral test on day 2. A typical TTC staining in FIG. 3A showed that pretreatment with Psilocybin reduced the infarct volume. To further demonstrate the topographic relationship of protection, the area of infarction in each 2-mm slice was compared in all animals receiving psilocybin and vehicle. Psilocybin significantly lowered infarction (p<0.001, two-way ANOVA). Post hoc NK test revealed that the significant difference mainly occurred on the rostral slices (p<0.05, FIG. 3B).

### Post-Stroke Treatment With Psilocybin Improves Behavior

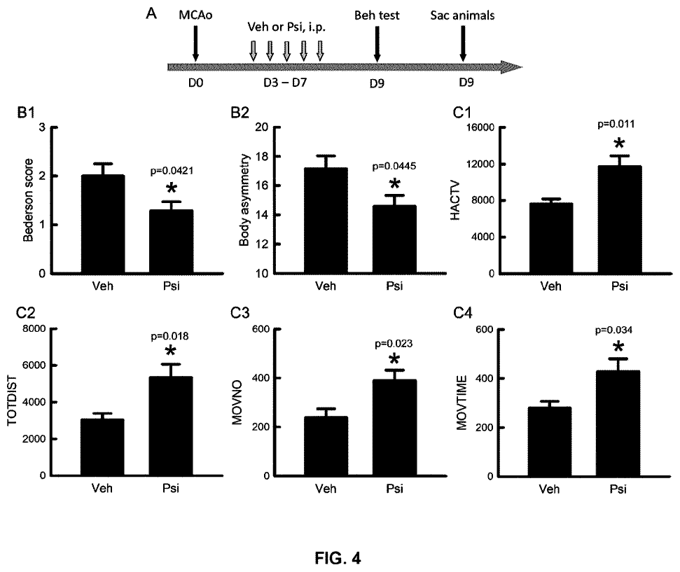

The effectiveness of post-stroke psilocybin therapy was examined in 13 stroke rats. Psilocybin (1 mg/kg/d, n=7) or vehicle (n=6) was delivered systemically (i.p.) from days 3 to 7 after the MCAo. Neurological and locomotor tests were examined on day 9 (FIG. 4A). Psilocybin significantly reduced neurological deficits (FIG. 4-B1, Bederson's neurological test, p=0.0421; B2: body asymmetry, p=0.0445, t-test) while increased locomotor behavior (FIG. 4-C1:horizontal activity p=0.011; C2: total distance traveled, p=0.018; C3: movement number, p=0.023; C4: movement time, p=0.034, t-test). These data suggest that early post-treatment with Psi improved behavior function in stroke rats.

### Post-Stroke Treatment With Psilocybin Reduced Brain Damage and Increased BDNF Expression

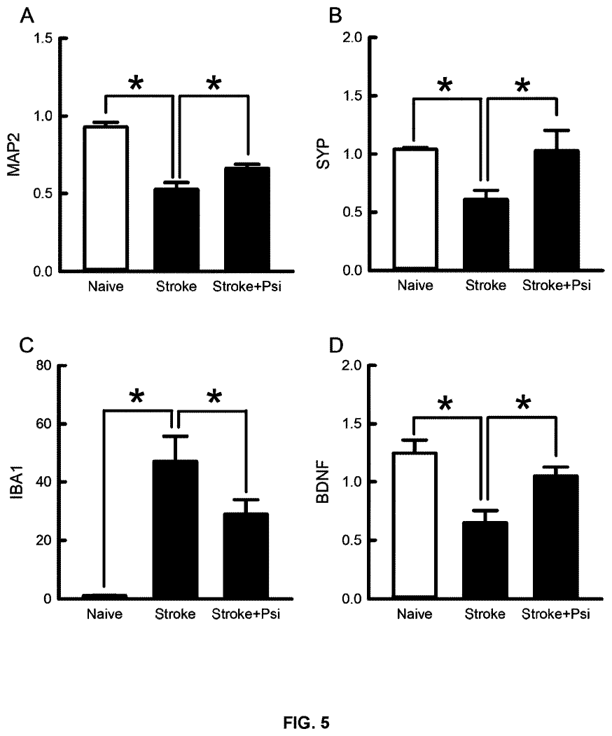

Animals receiving post-stroke psilocybin (1 mg/kg/d, n=7) or vehicle (n=6) treatment were sacrificed on day 9 after the MCAo. Additional 6 naive rats were used as control. Cerebral cortical tissue was collected for qRTPCR analysis. Ischemicinjury significantly reduced the expression of MAP2 and synaptophysin (FIG. 5A and B, p=0.013 and p=0.019, naive vs. stroke, one-way ANOVA+NK-test). Inflammatory marker IBA1 was significantly upregulated (p=0.037, naive vs. stroke, one-way ANOVA+NK-test, FIG. 5C). These responses were significantly reduced by Psilocybin (FIG. 5A-C). Psilocybin also significantly upregulated the expression of BDNF in the lesioned brain (FIG. 5D, p=0.01, stroke vs. stroke+PSI, one-way ANOVA+NK-test).

### ANA12 Antagonized Psilocybin-Mediated Neuroprotection in Vitro

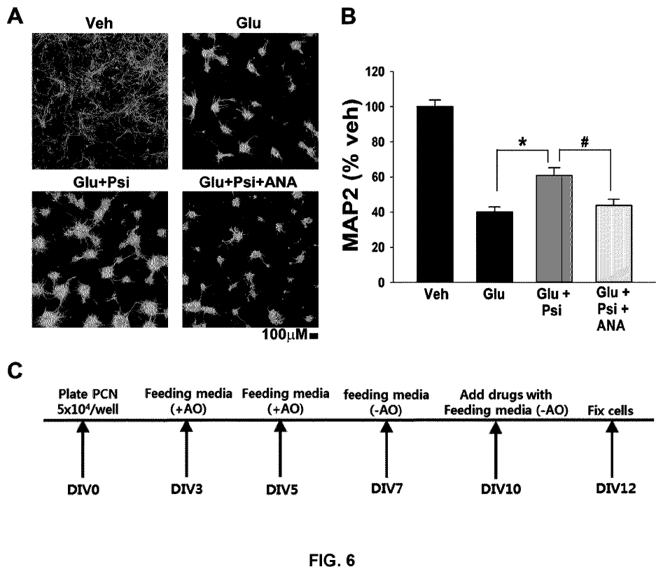

Since Psilocybin upregulated BDNF expression in the lesioned cortex, we next examined if the protective response of psilocybin involves BDNF in primary cortical neuronal culture. A selective BDNF antagonist ANA12 was included on D10 along with psilocybin and glutamate (see timeline in FIG. 6C). Co-treatment with ANA12 significantly antagonized psilocybin-mediated improvement in MAP2-ir (FIG. 6A and B, p<0.05, one-way ANOVA).

### ANA12 Antagonized Psilocybin-Mediated Neuroprotection in Vivo

The interaction of ANA12 and psilocybin was examined in 14 stroke rats. Psilocybin was given (i.c.v., 100 μM×20 μl) at 15 min prior to the MCAo (see Timeline, FIG. 7A). ANA12 was delivered intranasally after the MCAo on days 0 and 1 (100 μM×20 μL/d, n=7). Control stroke animals received intranasal saline administration (20 μL/d, n=7). ANA12 significantly antagonized psilocybin-mediated reduction in brain infarction (FIG. 7B) and neurological behavior (FIG. 7C1, C2).

Psilocybin-mediated improvements in horizontal activity and total distance traveled were significantly antagonized by ANA12 (FIG. 7D1-D2). These data suggest that BDNF is involved in psilocybin-induced protection.

### REFERENCES

1. WASSON R. G. The wondrous mushroom: mycolatry in Mesoamerica New York: McGraw-Hill; 1980.
2. LI N. X., HUY. R., CHEN W. N., ZHANG B. Dose effect of psilocybin on primary and secondary depression: a preliminary systematic review and meta-analysis, J Affect Disord 2022: 296: 26-34.
3. YU C. L., YANG F. C., YANG S. N., TSENG P. T., STUBBS B., YEH T. C. et al. Psilocybin for End-of-Life Anxiety Symptoms: A Systematic Review and Meta-Analysis, Psychiatry Investig 2021: 18: 958-967.
4. CARHART-HARRIS R., GIRIBALDI B., WATTS R., BAKER-JONES M., MURPHY-BEINER A., MURPHY R. et al. Trial of Psilocybin versus Escitalopram for Depression, N Engl J Med 2021: 384: 1402-1411.
5. JOHNSON M. W., GRIFFITHS R. R. Potential Therapeutic Effects of Psilocybin, Neurotherapeutics 2017: 14: 734-740.
6. PASSIE T., SEIFERT J., SCHNEIDER U., EMRICH H. M. The pharmacology of psilocybin, Addict Biol 2002: 7: 357-364.
7. HASLER F., BOURQUIN D., BRENNEISEN R., BAR T., VOLLENWEIDER F. X. Determination of psilocin and 4-hydroxyindole-3-acetic acid in plasma by HPLC-ECD and pharmacokinetic profiles of oral and intravenous psilocybin in man, Pharm Acta Helv 1997: 72: 175-184.
8. GEIGER H. A., WURST M. G., DANIELS R. N. DARK Classics in Chemical Neuroscience: Psilocybin, ACS Chem Neurosci 2018: 9: 2438-2447.
9. VOLLENWEIDER F. X., VOLLENWEIDER-SCHERPENHUYZEN M. F., BABLER A., VOGEL H., HELL D. Psilocybin induces schizophrenia-like psychosis in humans via a serotonin-2 agonist action, Neuroreport 1998: 9: 3897-3902.
10. CAMERON L. P., BENSON C. J., DUNLAP L. E., OLSON D. E. Effects of N, N-Dimethyltryptamine on Rat Behaviors Relevant to Anxiety and Depression, ACS Chem Neurosci 2018: 9: 1582-1590.
11. BECKER A. M., HOLZE F., GRANDINETTI T., KLAIBER A., TOEDTLI V. E., KOLACZYNSKA K. E. et al. Acute Effects of Psilocybin After Escitalopram or Placebo Pretreatment in a Randomized, Double-Blind, Placebo-Controlled, Crossover Study in Healthy Subjects, Clin Pharmacol Ther 2022: 111: 886-895.
12. LY C., GREB A. C., CAMERON L. P., WONG J. M., BARRAGAN E. V., WILSON P. C. et al. Psychedelics Promote Structural and Functional Neural Plasticity, Cell Rep 2018: 23: 3170-3182.
13. RAVAL N. R., JOHANSEN A., DONOVAN L. L., ROS N. F., OZENNE B., HANSEN H. D. et al. A Single Dose of Psilocybin Increases Synaptic Density and Decreases 5-HT2A Receptor Density in the Pig Brain, Int J Mol Sci 2021: 22.
14. DOSS M. K., POVAZAN M., ROSENBERG M. D., SEPEDA N. D., DAVIS A. K., FINAN P. H. et al. Psilocybin therapy increases cognitive and neural flexibility in patients with major depressive disorder, Transl Psychiatry 2021: 11: 574.
15. ALEKSANDROVA L. R., PHILLIPS A. G. Neuroplasticity as a convergent mechanism of ketamine and classical psychedelics, Trends Pharmacol Sci 2021: 42: 929-942.
16. HESSELGRAVE N., TROPPOLI T. A., WULFF A. B., COLE A. B., THOMPSON S. M. Harnessing psilocybin: antidepressant-like behavioral and synaptic actions of psilocybin are independent of 5-HT2R activation in mice, Proc Natl Acad Sci USA 2021: 118.
17. ODLAND A. U., KRISTENSEN J. L., ANDREASEN J. T. Investigating the role of 5-HT2A and 5-HT2C receptor activation in the effects of psilocybin, DOI, and citalopram on marble burying in mice, Behav Brain Res 2021: 401: 113093.
18. NARDAI S., LASZLO M., SZABO A., ALPAR A., HANICS J., ZAHOLA P. et al. N,N-dimethyltryptamine reduces infarct size and improves functional recovery following transient focal brain ischemia in rats, Exp Neurol 2020: 327: 113245.
19. YU S. J., WU K. J., BAE E., WANG Y. S., CHIANG C. W., KUO L. W. et al. Post-treatment with Posiphen Reduces Endoplasmic Reticulum Stress and Neurodegeneration in Stroke Brain, iScience 2020: 23: 100866.
20. SHEN H., CHEN G. J., HARVEY B. K., BICKFORD P. C., WANG Y. Inosine reduces ischemic brain injury in rats, Stroke 2005: 36: 654-659.

21. GLOBUS M. Y., ALONSO O., DIETRICH W. D., BUSTO R., GINSBERG M. D. Glutamate release and free radical production following brain injury: effects of posttraumatic hypothermia, J Neurochem 1995: 65: 1704-1711.
22. DINIS-OLIVEIRA R. J. Metabolism of psilocybin and psilocin: clinical and forensic toxicological relevance, Drug Metab Rev 2017: 49: 84-91.
23. LEE Y., LEE S. R., CHOI S. S., YEO H. G., CHANG K. T., LEE H. J. Therapeutically targeting neuroinflammation and microglia after acute ischemic stroke, BiomedResInt 2014: 2014: 297241.
24. ANTTILA J. E., ALBERT K., WIRES E. S., MATLIK K., LORAM L. C., WATKINS L. R. et al. Post-stroke Intranasal (+)-Naloxone Delivery Reduces Microglial Activation and Improves Behavioral Recovery from Ischemic Injury, eNeuro 2018: 5.
25. YU S. J., WU K. J., WANG Y. S., SONG J. S., WU C. H., JAN J. J. et al. Protective Effect of CXCR4 Antagonist CX807 in a Rat Model of Hemorrhagic Stroke, Int J Mol Sci 2020: 21.
26. NKADIMENG S. M., NABATANZI A., STEINMANN C. M. L., ELOFF J. N. Phytochemical, Cytotoxicity, Antioxidant and Anti-Inflammatory Effects of Psilocybe Natalensis Magic Mushroom, Plants (Basel) 2020: 9.
27. NKADIMENG S. M., STEINMANN C. M. L., ELOFF J. N. Anti-Inflammatory Effects of Four Psilocybin-Containing Magic Mushroom Water Extracts in vitro 15-Lipoxygenase on Activity and on Lipopolysaccharide-Induced Cyclooxygenase-2 and Inflammatory Cytokines in Human U937 Macrophage Cells, J Inflamm Res 2021: 14: 3729-3738.
28. FERRER I., BALLABRIGA J., MARTI E., PEREZ E., ALBERCH J., ARENAS E. BDNF up-regulates TrkB protein and prevents the death of CA1 neurons following transient forebrain ischemia, Brain Pathol 1998: 8: 253-261.
29. YU S. J., TSENG K. Y., SHEN H., HARVEY B. K., AIRAVAARA M., WANG Y. Local administration of AAV-BDNF to subventricular zone induces functional recovery in stroke rats, PLoS One 2013: 8: e81750.
30. AZOGU I., PLAMONDON H. Inhibition of TrkB at the nucleus accumbens, using ANA-12, regulates basal and stress-induced orexin A expression within the mesolimbic system and affects anxiety, sociability and motivation, Neuropharmacology 2017: 125: 129-145.
31. JORGENSEN T. S., WIUM-ANDERSEN I. K., WIUM-ANDERSEN M. K., JORGENSEN M. B., PRESCOTT E., MAARTENSSON S. et al. Incidence of Depression After Stroke, and Associated Risk Factors and Mortality Outcomes, in a Large Cohort of Danish Patients, JAMA Psychiatry 2016: 73: 1032-1040.
32. STEWART J. C., HAWKINS M. A., KHAMBATY T., PERKINS A. J., CALLAHAN C. M. Depression and Anxiety Screens as Predictors of 8-Year Incidence of Myocardial Infarction and Stroke in Primary Care Patients, Psychosom Med 2016: 78: 593-601.
33. LUO Y., KUO C. C., SHEN H., CHOU J., GREIG N. H., HOFFER B. J. et al. Delayed treatment with a p53 inhibitor enhances recovery in stroke brain, Ann Neurol 2009: 65: 520-530.
34. BORLONGAN C. V., TAJIMA Y., TROJANOWSKI J. Q., LEE V. M., SANBERG P. R. Transplantation of cryopreserved human embryonal carcinoma-derived neurons (NT2N cells) promotes functional recovery in ischemic rats, ExpNeurol 1998: 149: 310-321.
35. BEDERSON J. B., PITTS L. H., TSUJI M., NISHIMURA M. C., DAVIS R. L., BARTKOWSKI H. Rat middle cerebral artery occlusion: evaluation of the model and development of a neurologic examination, Stroke 1986: 17: 472-476.
36. LIU H. S., SHEN H., HARVEY B. K., CASTILLO P., LU H., YANG Y. et al. Post-treatment with amphetamine enhances reinnervation of the ipsilateral side cortex in stroke rats, Neuroimage 2011: 56: 280-289.
37. SHEN H., WANG Y. Correlation of locomotor activity and brain infarction in rats with transient focal ischemia, J Neurosci Methods 2010: 186: 150-154.

I claim:

1. A method for the treatment of stroke in a mammal comprising administering a therapeutically effective amount of psilocybin or a pharmaceutically acceptable salt or solvate thereof, together with one or more pharmaceutically acceptable carriers, diluents and excipients to a mammal in need thereof.

2. The method of claim 1, wherein the mammal is a human.

3. The method of claim 1, wherein the therapeutically effective amount is a dose within the range of about 0.5 mL/day to about 3 mL/day at about 0.5 mM to about 3.0 mM concentration.

4. The method of claim 1, wherein the therapeutically effective amount is a dose of about 0.5 mL/day at about a 2.0 mM concentration.

5. The method of claim 1, wherein the therapeutically effective amount is a dose of about 1.2 mL/day at about a 2.0 mM concentration.

6. The method of claim 1, wherein the therapeutically effective amount is a dose of about 3.0 mL/day at about a 2.0 mM concentration.

7. The method of any one of claims 1 to 6, wherein the stroke is ischemic brain injury.

8. The method of any one of claims 1 to 6, wherein the step of administering includes administering the psilocybin or a pharmaceutically acceptable salt or solvate thereof, together with one or more pharmaceutically acceptable carriers, diluents and excipients pre-or early post-stroke.

9. The method of any one of claims 1 to 8, wherein the step of administering includes administering the psilocybin or a pharmaceutically acceptable salt or solvate thereof, together with one or more pharmaceutically acceptable carriers, diluents and excipients, administering to the patient orally.

10. The method of claim 9, wherein the step of administering to the patient orally includes administering buccally or sublingually.

11. The method of claim 10, wherein the step of administering to the patient orally includes administering in as tablets, capsules, powders, granules, lozenges, creams or liquid preparations.

12. Use of a pharmaceutical composition including psilocybin or a pharmaceutically acceptable salt or solvate thereof, together with one or more pharmaceutically acceptable carriers, diluents and excipients for the treatment of stroke in a mammal.

13. The use of claim 12, wherein the mammal is a human.

14. The use of claim 13, wherein the therapeutically effective amount is a dose within the range of about 0.5 mL/day to about 3.0 mL/day at about 0.5 mM to about 3.0 mM concentration.

15. The use of claim 13, wherein the therapeutically effective amount is a dose of about 0.5 mL/day at about a 2.0 mM concentration.

16. The use of claim 13, wherein the therapeutically effective amount is a dose of about 1.2 mL/day at about a 2.0 mM concentration.

17. The use of claim 13, wherein the therapeutically effective amount is a dose of about 3.0 mL/day at about a 2.0 mM concentration.

18. The use of any one of claims 13 to 17, wherein the brain injury is selected from the group consisting of a mild brain injury or traumatic brain injury.

19. The use of claim 18, wherein the wherein the stroke is ischemic brain injury.

20. The use of any one of claims 13 to 19, wherein the composition is included in a carrier selected from the group consisting of tablets, capsules, powders, granules, lozenges, creams and liquid preparations.
